(19) United States
(12) Patent Application Publication
(10) Pub. No.: US 2022/0226232 A1
(43) Pub. Date: Jul. 21, 2022

(54) Dental formulation comprising xylitol and propolis

(71) Applicant: SERVIDENCE INC., Saint-Hyacinthe (CA)

(72) Inventor: Jacques Veronneau, Saint-Hyacinthe (CA)

(21) Appl. No.: 17/648,608

(22) Filed: Jan. 21, 2022

Related U.S. Application Data
(60) Provisional application No. 63/139,970, filed on Jan. 21, 2021.

(51) Int. Cl. A61K 8/98; A61Q 11/00; A61K 8/34

(52) U.S. Cl. CPC A61K 8/988; A61K 8/345; A61Q 11/00

(57) ABSTRACT

The present invention provides a dental formulation for the prevention and treatment of dental caries and dental hypersensitivity. The dental formulation is a toothpaste formulation comprising 25% of xylitol and 0.5% of propolis.

### CROSS-REFERENCE TO RELATED APPLICATIONS

The present patent application claims the benefits of priority of U.S. Patent Application No. 63/139,970, entitled “Dental formulation comprising xylitol and propolis” and filed at the United States Patent and Trademark Office on Jan. 21, 2021, the content of which is incorporated herein by reference.

### FIELD OF THE INVENTION

The present invention generally relates to dental formulations, more precisely to toothpaste for the prevention and/or treatment of dental carries and sensitivity.

### BACKGROUND OF THE INVENTION

Even with the declining prevalence of dental caries amongst industrialized countries during the past decades, explained mainly by the use of fluoride in various forms, dental caries is still a major problem in most industrialized countries, affecting from 60% to 90% of schoolchildren, and affecting the vast majority of adults. There is also a mounting international evidence that this decline is coming to an end and that many dental patients are still highly infected by cariogenic bacteria (100,000 Streptococcus mutans/mL of saliva). Given all of the publicly available evidences, caries still remains a significant health problem and is still the predominant reason for tooth loss in almost all age groups. Although dental caries is a largely preventable disease, untreated cavitated carious lesions in permanent teeth is still the most prevalent health condition in the world. In primary teeth, it represents one tenth of the most prevalent condition, but with the possibility of many dental, medical, social, and quality of life sequels. At the same time, real effectiveness of curative and preventive approaches on high risk caries patients are questioned in many countries.

Xylitol was discovered in 1890 by Fisher and Stahe in Germany as a sugar-alcohol and then, used as natural sugar subtract during the Second World War in Finland. We can find it naturally in the human body due to between five to fifteen grams of daily production. On a commercial scale, xylitol can be produced by chemical and biotechnological processes mainly from birch trees or corn cobs. Industrial xylitol production reproduces the same molecular structure of the natural one. Two hypothesis are leading the bacterial effect of xylitol on caries process: either a chewing movement, related to xylitol, producing saliva or as a sweetened replacer which is essentially not fermentable by the caries-inductive oral microflora. Today, xylitol's principal adopted mechanism is its earlier action in caries process and by so, is the prevention of carious lesions. Xylitol has been showed by several trials and confirmed by literature reviews as an antibacterial agent. Xylitol has also been presented recently as a potential therapeutic agent on caries. Therapeutic effects were estimated from xylitol trials using menthols, candies, syrup (8 grams/day), wipes and milk with success. At the end of the 1990's, two trials used toothpaste as vehicle for xylitol 10% (0.1 g/tooth brushing); one trial combined it to 0.243 NaF % and in the other, combined it to Sodium of Monofluorophaste % 1 100 PM. The trials combined had a total of 4 800 schoolchildren and reported a negligible prevention fraction of 10% and 13% respectively. The authors concluded that xylitol in toothpaste might be irrelevant.

The inventor was associated, as a lead researcher, to series of designs that generated scientific original data on an existing 25% xylitol toothpaste. A two years trial using 201 6-month-old children and their parents, showed a preventive fraction of 60% with this toothpaste compared to fluoride toothpaste. In addition, tested children reported 80% less cavitated lesions and 41% more initial lesions remineralization than the controlled children. The tested children also had their saliva collected at the beginning and end of the trial and a significant antibacterial effect was observed on cariogenic bacteria. This natural toothpaste was confirmed, for the first time, as an anti-caries agent. In addition, an observational test which followed 43 adults at a high risk of caries brushing their teeth for three months confirmed the huge antibacterial effect on cariogenic bacteria. Another observational test, with children, confirmed the sustainable and flora protective antibacterial effect of this toothpaste for six months minimally. Another benefit was demonstrated by this toothpaste: it seems that this formulation increases the incapacity of saliva compound. Furthermore, Cochrane reviews showed that an oral exposition of xylitol can leads to the prevention of otitis 20%. Finally, nine adults with a high risk of periodontal brushed their teeth for three months with this formulation and had their saliva collected pre and post brushing time frame. The formulation decreased high risk periodontal bacteria by 78% and middle risk periodontal bacteria by 68%, said bacteria being associated to lung cancer in population studies. Given all the previous now known benefits, this toothpaste is now sold in a few countries.

Dentinal hypersensitivity (DH) is a very frequently encountered clinical problem in all age groups, except for the very young. It has been reported that the mean prevalence of sensitive teeth is around 33%. The incidence of DH tends to peak among aging populations. With the population living and keeping their teeth longer, hypersensitivity, can be expected to become a growing concern because of the increased number of teeth with abrasion, erosion and attrition. This increasing part of the population having dentinal hypersensitivity symptoms cannot adopt the 25% xylitol toothpaste due to the absence of desensitizing effect in the existing formulation.

There is thus a need for a dental formulation comprising both xylitol and a desensitizing agent for the prevention and treatment of dental caries and DH.

### SUMMARY OF THE INVENTION

The aforesaid and other objectives of the present invention are realized by generally providing a dental formulation for the prevention and treatment of dental caries and dentinal hypersensitivity, the formulation comprising propolis as a first active agent and xylitol as a second active agent. The propolis proportion may be from about 0.25 wt. % to about 2.00 wt. %. In a further aspect of the invention, the propolis proportion is about 0.50 wt. %. The formulation may further comprise at least a carrying agent, a moisturizing agent, a thickening agent, an abrasive agent, a flavor agent, a surfactant agent and a foaming agent.

In another aspect of the invention, the formulation comprising water, barbadensis leaf juice, propolis, hydroxyethyl cellulose, magnesium aluminum silicate, calcium carbonate, lemon flavor, sodium bicarbonate and sodium lauryl sulfoacetate.

In yet another aspect of the invention, the xylitol proportion is from about 5 wt. % to about 25 wt. %. The xylitol proportion is about 5 wt. %. in another embodiment wherein it is about 25 wt. % in yet another embodiment.

In another aspect of the invention, the toothpaste formulation for treating dentinal hyper-sensitivity comprises the following raw materials:
about 19.83 wt. % of water;
about 43 wt. % of sorbitol;
about 19 wt. % of hydrated silica;
about 7 wt. % of glycerin;
about 5 wt. % of xylitol;
about 2 wt. % of propolis;
about 1.178 wt. % of sodium C14-16 olefin sulfonate;
about 1 wt. % of CI 77891;
about 0.8 wt. % of cellulose gum;
about 0.5 wt. % of aroma;
about 0.3 wt. % of xanthan gum;
about 0.2 wt. % of sodium saccharin;
about 0.17 wt. % of sodium benzoate; and
about 0.02174 wt. % of limonene.

In yet another aspect of the invention, the toothpaste formulation for treating dentinal hyper-sensitivity comprises the following raw materials:
about 34.42 wt. % of water;
about 25 wt. % of xylitol;
about 17.5 wt. % of hydrated silica;
about 17.36 wt. % of sorbitol;
about 1.75 wt. % of glycerin;
about 1 wt. % of CI 77891;
about 0.8 wt. % of cellulose gum;
about 0.527 wt. % of sodium lauroyl sarcosinate;
about 0.5 wt. % of aroma;
about 0.5 wt. % of propolis;
about 0.3 wt. % of xanthan gum;
about 0.17 wt. % of sodium benzoate;
about 0.13 wt. % of calcium glycerophosphate;
about 0.02 wt. % of sodium saccharin; and
about 0.02174 wt. % of limonene.

The features of the present invention which are believed to be novel are set forth with particularity in the appended claims.

### DETAILED DESCRIPTION OF THE PREFERRED EMBODIMENT

A novel dental formulation comprising xylitol and propolis will be described hereinafter. Although the invention is described in terms of specific illustrative embodiment(s), it is to be understood that the embodiment(s) described herein are by way of example only and that the scope of the invention is not intended to be limited thereby.

As used herein % or wt. % means % by weight as compared to the total weight percent of the phase or composition that is being discussed unless otherwise indicated.

By “about”, it is meant that the value of weight % can vary within a certain range depending on the margin of error of the method or device used to evaluate such weight %. A margin of error of 10% is generally accepted.

A total of three different propolis concentrations were first prepared. Five patients using a commercial toothpaste (mostly Sensodyne®) against DH were then recruited. They were asked to stop the use of the commercial toothpaste until the DH symptoms were back. Each patient was then assigned a propolis formulation to use; the exact propolis concentration being unknown to them and randomly allocated. They were asked to qualify the propolis exposure compared to their actual commercial toothpaste against DH. They were then asked to assess the pain from a scale of 1 to 10, with 10 being no pain, the effect using the responsiveness strategy. The new toothpaste was tested for three weeks. The first five patients were exposed to one out of three propolis concentrations at a time: 0.25%, 0.50% and 0.75%. Each patient was exposed to all three concentrations. The concentrations of 0.25 and 0.75 had no reported effect. The formulation with propolis at 0.50% had a significant effect, between 7 and 8 on the pain scale, and was judged as having more impact for the treatment of DH by patients compared to their commercial one. Surprisingly, no dose dependent response was observed.

Given previous studies outcome related to formulations comprising 25% xylitol and to the new results of the formulation study comprising propolis at 0.5%, both against caries and DH symptoms, the potential synergy and complementarity of both agents now has ground to be tested. No other publications documented this specific benefice of mixing 25% of xylitol to 0.5% of propolis in a dental formulation to combine the anti-caries and anti-sensitivity properties.

In order to test the obtained formulation, an observational test with cross-over treatments was performed. Ten patients, with the intention to use toothpaste comprising 25% xylitol, were complaining about the absence of anti-sensitive agents in the formulation. They all had a history with DH symptoms and were voluntarily recruited from two Quebec dental clinics. Having a desensitizing agent was a criteria for the treatment of DH with their current commercial toothpaste. All participants were evaluated using a scale from 0 to 10, in which 0 indicated no effect and 10 indicating an optimal effect. The majority of them reported the use of a commercially available Sensodyne® from Glaxo Smith Kline™. Their actual satisfaction with their commercially available toothpaste generated an average score of 7.0 out of 10. Participants were then asked to brush twice a day with the formulation samples of 25% xylitol and 0.5% propolis. Results showed that a positive effect was perceived within 3 days on average. On average, the participants gave a score of 8.0 out of 10, which firmly proves that the formulation provides benefits against both dental caries and DH. Furthermore, all recruited patients confirmed their interested to adopt this toothpaste in the future.

Another trial with five other patients and a similar approach was also conducted. In this trial, each patient was exposed to four concentrations of propolis: 1.00%, 1.25%, 1.50% and 2.00%. The trial lasted for 2 months before the results were compiled. This time, the results were the most promising with a concentration of 2.00% which resulted in a rating average of 7.7 out of 10.

These results support the effectiveness of a propolis-based toothpaste. More so, those results prove that simply increasing the propolis concentration does not automatically equate to increased positive results. Increasing the concentration from 0.5% to 0.75% did, in fact, diminish the positive impacts of the formulation. It is therefore important to have a specific concentration of propolis in a given formulation that was pre-emptively tested so as to ensure desired performance. Furthermore, most patients were interested in replacing their present commercially available toothpaste with a natural one having propolis as the active agent against DH.

Example 1 provides a representative toothpaste formulation of the invention which comprises 2% of propolis as the only active agent against DH.

| INGREDIENTS | % wt |
|---|---|
| Water | 19.83 |
| Sorbitol | 43 |
| Hydrated silica | 19 |
| Glycerin | 7 |
| Xylitol | 5 |
| Propolis | 2 |
| Sodium C14-16 olefin sulfonate | 1.178 |
| Cl 77891 | 1 |
| Cellulose gum | 0.8 |
| Aroma | 0.5 |
| Xanthan gum | 0.3 |
| Sodium saccharin | 0.2 |
| Sodium benzoate | 0.17 |
| Limonene | 0.02174 |

In a further embodiment, the toothpaste formulation may comprise propolis and a high amount of xylitol. The formulation may comprise 0.5% or propolis and about 25% of xylitol. Xylitol is an ingredient that has proven its effectivity against cavities, especially in commercially available toothpastes such as Cari0®.

Example 2 provides a representative toothpaste formulation of the invention. The formulation comprises 0.5% of propolis and 25% of xylitol.

| INGREDIENTS | % wt |
|---|---|
| Water | 34.421 |
| Xylitol | 25 |
| Hydrated silica | 17.5 |
| Sorbitol | 17.36 |
| Glycerin | 1.75 |
| Cl 77891 | 1 |
| Cellulose gum | 0.8 |
| Sodium lauroyl sarcosinate | 0.527 |
| Aroma | 0.5 |
| Propolis | 0.5 |
| Xanthan gum | 0.3 |
| Sodium benzoate | 0.17 |
| Calcium glycerophosphate | 0.13 |
| Sodium saccharin | 0.02 |
| Limonene | 0.02174 |

Furthermore, the tested toothpaste formulations had the following characteristics. Tested with a PHmetry test diluted at 25%, the pH value of each formulation varied between 6 to 8. A densimeter test revealed a density of about 1.35 g/mL for each formulation. Both tests of aerobic mesophilic flora and moulds and yeasts resulted in under 100 Ufc/g as per rapid microbiological method and/or UNE/EN/ISO 21149:2017 and UNE/EN/ISO 16212:2017. More so, there was an absence of pathogenic microorganisms in the formulations from the tests of absence/presence, rapid microbiological method and/or UNE/EN/ISO 21150:2016, UNE/EN/ISO 22717:2016, UNE/EN/ISO 22718:2016 and UNE/EN/ISO 18416:2016. Additionally, each toothpaste formulation had the following characteristics based on sensorial testing methods: they had a pasty texture, their color was a slightly yellowish white and they tasted like sweet mint. These characteristics are further proof that the different formulations, representing different aspects of the invention, are well suited for at-home usage.

While illustrative and presently preferred embodiment(s) of the invention have been described in detail hereinabove, it is to be understood that the inventive concepts may be otherwise variously embodied and employed and that the appended claims are intended to be construed to include such variations except insofar as limited by the prior art.

1) A dental formulation for the prevention and treatment of dentinal hypersensitivity, and dental caries, the formulation comprising propolis as a first active agent and xylitol as a second active agent.

2) The dental formulation of claim 1, wherein the propolis proportion is from about 0.25 wt. % to about 2.00 wt. %.

3) The dental formulation of claim 2, wherein the propolis proportion is about 0.50 wt. %.

4) The dental formulation of claim 1, the formulation further comprising at least a carrying agent, a moisturizing agent, a thickening agent, an abrasive agent, a flavor agent, a surfactant agent and a foaming agent.

5) The dental formulation of claim 4, the formulation comprising water, barbadensis leaf juice, propolis, hydroxyethyl cellulose, magnesium aluminum silicate, calcium carbonate, lemon flavor, sodium bicarbonate and sodium lauryl sulfoacetate.

6) The dental formulation of claim 1, wherein the xylitol proportion is from about 5 wt. % to about 25 wt. %.

7) The dental formulation of claim 6, wherein the xylitol proportion is about 5 wt. %.

8) The dental formulation of claim 6, wherein the xylitol proportion is about 25 wt. %.

9) A dental formulation for the prevention and treatment of dental caries and dentinal hypersensitivity, comprising:
about 19.83 wt. % of water;
about 43 wt. % of sorbitol;
about 19 wt. % of hydrated silica;
about 7 wt. % of glycerin;
about 5 wt. % of xylitol;
about 2 wt. % of propolis;
about 1.178 wt. % of sodium C14-16 olefin sulfonate;
about 1 wt. % of CI 77891;
about 0.8 wt. % of cellulose gum;
about 0.5 wt. % of aroma;
about 0.3 wt. % of xanthan gum;
about 0.2 wt. % of sodium saccharin;
about 0.17 wt. % of sodium benzoate; and
about 0.02174 wt. % of limonene.

10) A dental formulation for the prevention and treatment of dental caries and dentinal hypersensitivity, comprising:
about 34.42 wt. % of water;
about 25 wt. % of xylitol;
about 17.5 wt. % of hydrated silica;
about 17.36 wt. % of sorbitol;
about 1.75 wt. % of glycerin;
about 1 wt. % of CI 77891;
about 0.8 wt. % of cellulose gum;
about 0.527 wt. % of sodium lauroyl sarcosinate;
about 0.5 wt. % of aroma;
about 0.5 wt. % of propolis;
about 0.3 wt. % of xanthan gum;
about 0.17 wt. % of sodium benzoate;
about 0.13 wt. % of calcium glycerophosphate;
about 0.02 wt. % of sodium saccharin; and
about 0.02174 wt. % of limonene.
